(19) United States
(12) Patent Application Publication
(10) Pub. No.: US 2026/0265809 A1
(43) Pub. Date: Sep. 10, 2026

(54) QUANTIFICATION OF FREE GUIDE RNA IN CRISPR COMPLEXES

(71) Applicant: ALDEVRON LLC, Fargo, ND (US)

(72) Inventors: JONATHAN W. COOPER, Fargo, ND (US); ALEXANDER STAATS, Fargo, ND (US); CHUENCHANOK KHODTHONG, Fargo, ND (US)

(21) Appl. No.: 19/164,943

(22) PCT Filed: Apr. 1, 2024
(86) PCT No.: PCT/US24/22511

§ 371 Date: Sep. 12, 2025

Related U.S. Application Data
(60) Provisional application No. 63/493,225, filed on Mar. 30, 2023.

(51) Int. Cl. C12Q 1/6806; C12N 9/22; C12N 15/113

(52) U.S. Cl. CPC C12Q 1/6806; C12N 9/222; C12N 15/113; C12N 2310/20

(57) ABSTRACT

The present invention relates to a novel analytical method for quantifying the concentration of uncomplexed (“free”) gRNA present in Cas-gRNA ribonucleoprotein complexes manufactured for genome editing.

### CROSS-REFERENCE TO RELATED APPLICATION

This application claims priority from U.S. Provisional Application 63/493,225, filed Mar. 30, 2023, the content of which is incorporated herein by reference in its entirety.

### BACKGROUND OF THE INVENTION

Gene editing is being developed as a therapeutic tool for treating a variety of diseases. One approach to gene editing involves the use of the CRISPR (Clustered Regularly Interspaced Short Palindromic Repeats) technology. This technology utilizes RNA-guided endonucleases such as Cas (CRISPR-associated) enzymes, to target specific genomic sequences that are complementary to the guide RNA. Guide RNA (gRNA) is a type of RNA molecule that binds to the Cas endonuclease and specifies, based on the targeting sequence of the gRNA, the location at which the Cas nuclease will cut the DNA strand. The most frequently used Cas endonuclease is Streptococcus pyogenes Cas9 (SpCas9). A Cas9 protein is comprised of a recognition (REC) lobe and a nuclease (NUC) lobe. The NUC lobe comprises a domain that interacts with the protospacer-adjacent motif (PAM) and two distinct nuclease domains, HNH and RuvC.

A Cas-gRNA ribonucleoprotein (RNP) is a complex between a Cas nuclease and one or more guide RNAs (gRNAs), and are used for gene editing therapeutics. When these RNPs are manufactured for intracellular delivery, it is required by the FDA to quantify the concentration of uncomplexed (“free”) gRNA present in these manufactured RNPs. However, there is currently nothing in the scientific literature describing a methodology to fulfill this regulatory need. Thus, there remains a need for accurate and reproducible analytical methods for determining the quantity of free gRNA present in Cas-gRNA RNP complexes manufactured for intracellular delivery.

### SUMMARY OF THE INVENTION

The present disclosure provides a method of quantifying by capillary zone electrophoresis (CZE) free RNA present in a composition comprising ribonucleoprotein (RNP) complexes formed between a gene-editing endonuclease and one or more associated RNAs. The method comprises the steps of (i) providing a CZE system comprising a source reservoir and a destination reservoir containing a separation buffer, wherein the source reservoir further contains an RNA-staining dye; an anode placed in the source reservoir and a cathode placed in the destination reservoir, wherein the anode and the cathode are connected to a power supply; a fused silica capillary tube connecting the two reservoirs; and a detector proximal to the cathode for detecting signals from the capillary tube; (ii) introducing the composition to the capillary tube proximal to the anode; (iii) performing electrophoresis on the system; and (iv) quantifying signal from RNA-bound dye by using the detector, wherein the quantity of the signal is indicative of the level of free RNA in the composition.

In some embodiments, the RNA is a guide RNA (gRNA), e.g., a gRNA that is at least 40 or at least 100 nucleotides in length. In some embodiments, the gene-editing endonuclease is a CRISPR endonuclease, e.g., Streptococcus pyogenes Cas9 (SpCas9), Acidaminococcus Cpf1, and Eureca-V. In some embodiments, the separation buffer, e.g., a borate buffer, is formulated to maintain the native conformation of the RNP. In some embodiments, the RNA-staining dye is a fluorescent dye, e.g., a dye comprising a chromophore unit whose chemical structure is shown below.

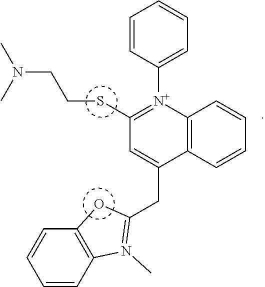

In some embodiments, detector is a laser-induced fluorescence (LIF) detector. In some embodiments, free RNA can be detected at a concentration as low as 0.2 μM or 6.8 μg/ml.

The present disclosure also provides a composition comprising a ribonucleoprotein (RNP) complex formed between a gene-editing endonuclease and one or more associated RNAs, said composition obtained by a process that separates any free RNA from the RNP complex, wherein the process comprises (i) providing a CZE system comprising a source reservoir and a destination reservoir containing a separation buffer, wherein the source reservoir further contains an RNA-staining dye; an anode placed in the source reservoir and a cathode placed in the destination reservoir, wherein the anode and the cathode are connected to a power supply; a fused silica capillary tube connecting the two reservoirs, and a detector proximal to the cathode for detecting signals from the capillary tube; (ii) introducing the composition to the capillary tube proximal to the anode; (iii) performing electrophoresis on the system to separate the free RNA from the RNP complex; and (iv) quantifying signal from RNA-bound dye by using the detector, wherein the quantity of the signal is indicative of the concentration of free RNA in the RNP complex.

In some embodiments, the RNA is a guide RNA (gRNA), e.g., a gRNA that is at least 40 or at least 100 nucleotides in length. In some embodiments, the gene-editing endonuclease is a CRISPR endonuclease, e.g., Streptococcus pyogenes Cas9 (SpCas9), Acidaminococcus Cpf1, and Eureca-V. In some embodiments, the separation buffer, e.g., a borate buffer, is formulated to maintain the native conformation of the RNP. In some embodiments, the RNA-staining dye is a fluorescent dye, e.g., a dye comprising a chromophore unit whose chemical structure is shown below.

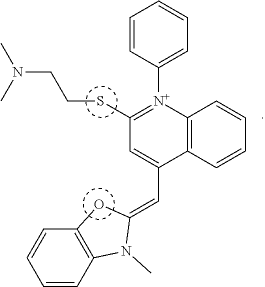 [SMILES: CN(C)CCSc1cc(/C=C2\Oc3ccccc3N2C)c2ccccc2[n+]1-c1ccccc1]

In some embodiments, detector is a laser-induced fluorescence (LIF) detector. In some embodiments, the concentration of free RNA is at least 0.2 μM or 6.8 μg/ml.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 an electropherogram showing a peak corresponding to a 100 nucleotide long uncomplexed gRNA. The migration time is shown on the X-axis and the fluorescence signal is shown on the Y-axis.

FIG. 2 is an electropherogram showing peaks corresponding to a 100 nucleotide long uncomplexed gRNA and a 100 nucleotide long gRNA complexed with a Cas9 protein (Cas9-gRNA ribonucleoprotein (RNP) complex). The migration times are shown on the X-axis and the fluorescence signal is shown on the Y-axis. The earlier peak corresponds to the Cas9-gRNA RNP complex and the later peak corresponds to the uncomplexed (“free”) gRNA.

FIGS. 3A and 3B are gRNA standard calibration curves. The gRNA concentrations (0.2, 0.4, 0.6, 0.8, and 1.0 μM) are shown on the X-axis and the peak area is shown on the Y-axis.

FIG. 4 is a chart showing the amount and percentage (%) of uncomplexed (“free”) gRNA present in an RNP complex of five concentrations (0.2, 0.4, 0.6, 0.8, and 1.0 μM). The RNP concentration (μM) is shown on the X-axis, percentage (%) of uncomplexed (“free”) gRNA present in an RNP complex (depicted as orange vertical bars) is shown on the right Y-axis, and the peak area of free gRNA (depicted as blue circles) is shown on the left Y-axis.

FIG. 5 is a chart showing the percentage (%) of uncomplexed (“free”) gRNA present two different RNP complexes. The first and second RNP complexes comprise gRNA and a CRISPR spCas9 endonuclease in a concentration ratio of 1:2 and 1:4, respectively. The gRNA concentration (μM) is shown on the X-axis, the peak area (depicted as orange vertical bars) is shown on the left Y-axis, and the % of free gRNA (depicted as yellow circles) is shown on the right Y-axis.

FIG. 6 is a chart showing the percentage (%) spike recovery of uncomplexed (“free”) gRNA present in an RNP sample that was spiked with the gRNA. Concentrations of RNP (0.4 μM), free gRNA (0.2, 0.4, 0.6, 0.8, or 1.0 μM), and (RNP+free gRNA) are shown on the X-axis; the peak area (depicted as blue vertical bars) is shown on the left Y-axis, and the % of spike recovery (depicted as orange circles) is shown on the right Y-axis. A standard gRNA calibration curve is shown on the top left corner of the chart.

FIGS. 7A, 7B, and 7C are gRNA standard calibration curves for runs 1-3, respectively, as described in Example 2. The gRNA concentrations (0.4, 0.8, 1.2, 1.6, 2.0, and 2.2 μM) are shown on the X-axis and the peak area is shown on the Y-axis.

FIG. 8 an electropherogram for the formulation buffer (negative control). The migration time is shown on the X-axis and the fluorescence signal is shown on the Y-axis.

FIG. 9 an electropherogram showing peaks corresponding to an uncomplexed gRNA and a Cas9-gRNA RNP complex. The migration times are shown on the X-axis and the fluorescence signal is shown on the Y-axis. The earlier peak corresponds to the gRNA bound to the RNP complex (“bound” gRNA) and the later peak corresponds to the uncomplexed (“free”) gRNA.

FIG. 10 is a chart showing the linearity plot of the expected vs. theoretical free gRNA content. Known amounts of gRNA were spiked into an RNP sample and the corresponding concentrations were measured. The expected result is plotted against the theoretical result. Observed gRNA concentrations (μM) are shown on the X-axis and expected gRNA concentrations (μM) are shown on the Y-axis.

FIG. 11 is a set of overlaid electropherograms showing peaks corresponding to uncomplexed gRNA and Cas9-gRNA RNP complex for RNP samples (each at a concentration of 0.3 μM) spiked with varying concentrations (0, 0.4, 0.6, 0.8, 1.0, and 1.2 μM) of gRNA. The migration times are shown on the X-axis and the fluorescence signal is shown on the Y-axis. The earlier set of peaks corresponds to the gRNA bound to the RNP complex (“bound” gRNA) and the later set of peaks corresponds to the uncomplexed (“free”) gRNA.

### DETAILED DESCRIPTION OF THE INVENTION

The present disclosure provides a novel capillary zone electrophoresis (CZE) assay for separating, detecting, and/or quantifying the concentration of free intact ribonucleic acid (RNA), e.g., guide RNA (gRNA), in a ribonucleoprotein (RNP) complex, e.g., a Cas9-gRNA RNP complex. “Free” RNA in an RNP complex refers to any RNA that is not bound to an RNA-binding protein (RBP), e.g., a Cas9 nuclease. In capillary zone electrophoresis, separation of molecules takes place inside a capillary tube filled with a separation buffer. Molecules are separated according to their charge-to-mass ratios. In the present disclosure, one or more intact RNA molecules are separated from any unbound RBP and the RNP complex based on their charge-to-mass ratios. The separated RNA molecules are then stained with a fluorescent dye and detected using a laser-induced fluorescence (LIF) detector as indicated by the time it takes for the RBP-free RNAs to migrate to the detection window. The separated RNAs appear as peaks in an electropherogram, which plots time versus detector signal, and the area under each peak is determined. The concentration of free intact RNA in the RNP sample is calculated using an RNA standard calibration curve and the peak areas of the free RNAs in the RNP sample.

The analytical method described in the present disclosure is advantageous because fluorescence detection of free RNA is linear, accurate, precise, repeatable, sensitive, and reproducible. This assay is suitable for separation, detection and/or quantification of free RNA present in RNP complexes comprising various ratios of RNAs and RBPs. This assay is also suitable for separation of RNA molecules of different sizes from a variety of RBPs.

Due to these improvements, the present analytical method will allow CZE-based quantification of free intact RNA to be widely employed in the biochemical analytical field for biopharmaceutical research, medical diagnosis, and environmental studies. This will be applicable for Cas9, Cas12a, and other nucleases or proteins that have different charge density compared to oligonucleotides within a separation buffer.

### I. Capillary Zone Electrophoresis

In capillary zone electrophoresis (CZE), also known as capillary electrophoresis (CE), target analytes are separated from other components in a mixture based on their charge-to-mass ratios under the influence of an electric field in a buffer-filled capillary (Sun et al., Proteomics (2014) 14(0):622-8). A typical CZE system comprises some or all of the following components:
(i) Capillary: a capillary tube is used for the separation of the target analyte from one or more contaminants.
(ii) Sample vial: a container into which a source sample is placed.
(iii) Injector: also known as a sample manager or auto sampler, an injector is used to introduce the source sample into the capillary tube.
(iv) Separation buffer: a buffer that allows the electrophoretic migration of the target analyte ions in an electric field.
(v) Source reservoir: a first reservoir at the injection end of the capillary tube containing the separation buffer and an anode.
(vi) Destination reservoir: a second reservoir at the detector end of the capillary tube containing the separation buffer and a cathode.
(vii) Power supply: a high-voltage power supply (e.g., 10 to 30 kV) to drive the separation of the target analyte.
(viii) Detector: a fluorescence detector, e.g., a laser-induced fluorescence (LIF) detector, or an UV detector.
(ix) Detection window: a region of capillary where the polyimide coating is absent.

### A. Capillary

In some embodiments, the capillary tube is a coated or uncoated fused silica capillary tube. Fused silica (fused silicon dioxide (SiO₂)) is non-crystalline glass form of silicon dioxide which is manufactured by melting crystalline silica. In some embodiments, the capillary tube is an uncoated (bare) fused silica capillary tube. In some embodiments, the capillary tube is about 10 to about 100 cm long and has an inner diameter of about 10 to about 100 m. In some embodiments, the capillary tube is about 67 cm long and has an inner diameter of about 50 m. In some embodiments, the capillary tube may be a narrower bore inner diameter capillary tube.

### B. Separation Buffer

In some embodiments, the separation buffer may contain ammonium acetate, ammonium formate, ammonium carbonate, ammonium bicarbonate, succinate, acetic acid, boric acid, borate, trifluoroacetic acid, formic acid, oxalic acid, phosphoric acid, iminodiacetic acid, aspartic acid, malonic acid, citric acid, citrate, succinic acid, MES (2-(N-morpholino) ethanesulfonic acid), ADA (N-2-acetamidoiminodiacetic acid), PIPES (piperazine-N,N′-bis(2-ethanesulfonic acid)), ACES (N-(2-acetamido)-2-aminoethanesulfonic acid), BIS-TRIS propane, imidazole, MOPS (3-(N-morpholino)propanesulfonic acid), MOPSO (2-Hydroxy-3-morpholinopropanesulfonic acid), Morpholine, TES (2-(tris(hydroxylmethyl)methyl)amino)ethanesulfonic acid), hydrochloride, HEPES (4-(2-hydroxyethyl)-1-piperazineethanesulfonic acid), HEPPS (4-(2-hydroxyethyl)-1-piperazineethanesulfonic acid), CHAPSO (cholamidopropyl]dimethylammonio)-2-hydroxy-1-propanesulfonate, CHES (2-(yclohexylamino)ethanesulfonic aci), CAPS (N-cyclohexyl-3-aminopropanesulfonic acid), hydrazine, TRICINE (N-tris(hydroxymethyl)methylglycine), TRIS (tris(hydroxymethyl)aminomethane), BICINE (bis(2-hydroxyethyl)amine), Glycine amide, glycylglycine, ammonia, trimethylamine, Imidazole, Phosphate, Succinate, and triethylamine. In some embodiments, the separation buffer contains boric acid.

In some embodiments, the separation buffer may contain one or more additives, such as salts, surfactants, chelators, glycerol, PEGs (polyethylene glycols), sucrose, glucose, amino acids, guanidine HCl, and urea. In some embodiments, the salt is selected from ammonium sulfate ((NH₄)₂SO₄) sodium chloride (NaCl), sodium citrate, sodium sulfate (Na₂SO₄), sodium tetraborate (Na₂B₄O₇), potassium chloride (KCl), calcium chloride (CaCl₂)), and magnesium chloride (MgCl₂), In some embodiments, the surfactant is selected from Polysorbate 80, Triton X-100, Tween-80, CHAPS, and sodium dodecyl sulfate (SDS). In some embodiments, the chelator is EDTA (ethylenediaminetetraacetic acid). In some embodiments, the amino acid is selected from glycine, proline, and L-arginine. In some embodiments, the PEG is PEG 3350.

### C. Fluorescent Dye

In some embodiments, the separation buffer may contain a fluorescent dye. In some embodiments, the fluorescent dye is a nucleic acid binding dye, e.g., an RNA-binding dye. In some embodiments, the fluorescent nucleic acid binding dye is selected from Invitrogen™ Molecular Probes™ SYTO™ RNASelect green-fluorescent cell stain, RiboGreen®, YOYO™_1, SYTOX™ Blue stain, and SYBR™ Green II RNA gel stain. In some embodiments, the fluorescent nucleic acid binding dye is SYBR™ Green II RNA gel stain and chemical structure of its chromophore unit (Saarnio et al., Dyes and Pigments (2020) 177:108282) is shown below:

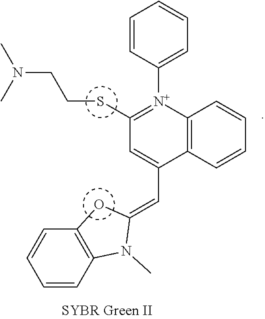 [SMILES: CN(C)CCSc1cc(/C=C2\Oc3ccccc3N2C)c2ccccc2[n+]1-c1ccccc1]

### II. Target Analyte

In some embodiments, the target analyte is a free intact RNA molecule. In some embodiments, the RNA molecule is greater than 10, 20, 30, 40, 50, 60, 70, 80, 90, 100, 150, or 200 nucleotides in length. In some embodiments, the RNA molecule is less than 50, 60, 70, 80, 90, 100, 150, or 200 nucleotides in length. In some embodiments, the RNA molecule is 10, 20, 30, 40, 50, 60, 70, 80, 90, 100, 110, 120, 130, 140, 150, 160, 170, 180, 190, or 200 nucleotides in length. In certain embodiments, the RNA molecule is at least 40 and no greater than 100 nucleotides in length.

An RNA molecule of the present disclosure may be naturally occurring or artificially designed. In some embodiments, the RNA molecule is a protein-coding mRNA molecule. In some embodiments, the RNA molecule is a non-protein-coding or non-coding RNA molecule (ncRNA). In some embodiments, the RNA molecule contained chemically modified nucleic acids. In some embodiments, the ncRNA is a long non-coding RNA (lncRNA; >200 nucleotides) or a short non-coding RNA (sncRNA; <200 nucleotides). In some embodiments, the ncRNA includes, but is not limited to, transfer RNA (tRNA), tRNA-derived small RNA (tsRNA), ribosomal RNA (rRNA), microRNA, short interfering RNA (siRNA), PIWI-interacting RNA (piRNA), small nuclear RNA (snRNA), small nucleolar RNA (snoRNA), snoRNA-derived small RNAs (sdRNAs), extracellular RNA (exRNA), small Cajal body-specific RNA (scaRNA), Xist, and HOTAIR.

### A. Guide RNA

In some embodiments, the RNA molecule, e.g., a guide RNA (gRNA) molecule, is associated with a gene-editing endonuclease. A gRNA molecule as described herein may comprise two parts: 1) a nucleotide sequence comprising a “targeting sequence” that is complementary to the RNA or DNA target site and may hybridize to the target site nucleic acid sequence, and 2) a nucleotide sequence that binds a polynucleotide guided RNA- or DNA-binding domain (e.g., a CRISPR-Cas protein domain). The target site may be a double-stranded DNA sequence comprising a PAM sequence as well as the target sequence, which is located on the same strand as, and directly adjacent to, the PAM sequence. The nucleotide sequence in 1) may be referred to as, e.g., a crispr RNA, or crRNA. The nucleotide sequence in 2) may be referred to as a scaffold sequence of a guide nucleic acid, e.g., a tracrRNA, or an activating region of a guide nucleic acid, and may comprise a stem-loop structure. Parts 1) and 2) as described above may be fused to form one single guide (e.g., a single guide RNA, or sgRNA), or may be on two separate nucleic acid molecules. In some embodiments, a gRNA molecule comprises parts 1) and 2) connected by a linker. In some embodiments, a gRNA molecule comprises parts 1) and 2) connected by a non-nucleic acid linker, for example, a peptide linker or a chemical linker.

An RNA molecule of the present disclosure may be single-stranded or double-stranded. The RNA may contain naturally occurring ribonucleotides, and/or chemically modified analogs thereof. As used herein, the term “RNA” is not limited to an oligoribonucleotide with only ribose-containing nucleotides. An RNA herein may encompass a polynucleotide molecule where the ribose moiety in some or all of its nucleotides has been replaced by another moiety. An RNA of the present disclosure may comprise one or more modifications. Modifications may include any modification known in the art, including, for example, end modifications, base modifications, sugar modifications/replacements, and backbone modifications. End modifications may include, for example, 5′ end modifications (e.g., phosphorylation, conjugation, and inverted linkages) and 3′ end modifications (e.g., conjugation, DNA nucleotides, and inverted linkages). Base modifications may include, e.g., replacement with stabilizing bases, destabilizing bases or bases that base-pair with an expanded repertoire of partners, removal of bases (abasic modifications of nucleotides), or conjugated bases. Sugar modifications or replacements may include, e.g., modifications at the 2′ or 4′ position of the sugar moiety, or replacement of the sugar moiety. Backbone modifications may include, for example, modification or replacement of the phosphodiester linkages, e.g., with one or more phosphorothioates, phosphorodithioates, phosphotriesters, methyl and other alkyl phosphonates, phosphinates, and phosphoramidates. Additional modifications may include conjugation of RNA with fluorescent dyes and other tracking molecules.

### B. RNA-Binding Protein (RBP) Complex

In some embodiments, the target analyte is separated from an uncomplexed RNA-binding protein (RBP). RBPs comprise one or more RNA-binding domains (RBDs). Exemplary RBDs include, but are not limited to, RNA Recognition Motifs (RRMs), K-homology domains (KHs), RGG (Arg-Gly-Gly) boxes, zinc fingers, double stranded RNA-binding domains (dsRBDs), target recognition (REC) lobes, Pumilio/PUF domains, and Piwi/Argonaute/Zwille (PAZ) domains. In some embodiments, the RBP is a gene-editing endonuclease, e.g., a CRISPR-associated (Cas) endonuclease. In some embodiments, the Cas endonuclease is a Cas3, Cas9, or Cas10 endonuclease. The Cas endonuclease can be enzymatically active or dead (dead Cas9 or dCas9). In some embodiments, the Cas endonuclease is a Cas12a (Cpf1) endonuclease. The Cpf1 endonuclease can be enzymatically active or dead (dead Cpf1 or dCpf1).

### C. Ribonucleoprotein (RNP) Complex

In some embodiments, the target analyte is separated from a ribonucleoprotein (RNP) complex, i.e., a complex of RNA and RNA-binding protein. In some embodiments, the RNP complex is a gRNA-CRISPR/Cas endonuclease RNP complex suitable for direct intracellular delivery. In some embodiments, the RNP complex is a gRNA-Cas3 RNP complex. In some embodiments, the RNP complex is a gRNA-Cas9 RNP complex. In some embodiments, the RNP complex is a gRNA-Cas10 RNP complex. In some embodiments, the RNP complex is a gRNA-Cas12 RNP complex. In some embodiments, the RNP complex is a gRNA-Eureca-V RNP complex.

In some embodiments, the gRNA is complexed with the RBP in a ratio greater than 1:1, 1:2, 1:3, 1:4, 1:5, 1:6, 1:7, 1:8, 1:9, or 1:10. In some embodiments, the gRNA is complexed with the RBP in a ratio less than 1:1, 1:2, 1:3, 1:4, 1:5, 1:6, 1:7, 1:8, 1:9, or 1:10. In some embodiments, the gRNA is complexed with the RBP in a ratio of 1:1, 1:2, 1:3, 1:4, 1:5, 1:6, 1:7, 1:8, 1:9, or 1:10.

### III. Applications

The novel CZE assay of the present disclosure can be used to analyze (e.g., detect and/or quantify) a variety of free intact RNA molecules, including gRNA molecules, in a sample of an RNP complex, e.g., a Cas3-gRNA RNP complex, a Cas9-gRNA RNP complex, a Cas10-gRNA RNP complex, a Cas12-gRNA RNP complex, and a Eureca-V-gRNA RNP complex. In some embodiments, the gRNA molecule is an sgRNA molecule. In some embodiments, the CZE assay described herein is used to quantify the concentration of free intact gRNA or sgRNA present in a Cas3-gRNA or Cas3-sgRNA RNP complex, respectively. In some embodiments, the CZE assay described herein is used to quantify the concentration of free intact gRNA or sgRNA present in a Cas9-gRNA or Cas9-sgRNA RNP complex, respectively. In some embodiments, the CZE assay described herein is used to quantify the concentration of free intact gRNA or sgRNA present in a Cas10-gRNA or Cas10-sgRNA RNP complex, respectively. In some embodiments, the CZE assay described herein is used to quantify the concentration of free intact gRNA or sgRNA present in a Cas12-gRNA or Cas12-sgRNA RNP complex, respectively. In some embodiments, the CZE assay described herein is used to quantify the concentration of free intact gRNA or sgRNA present in a Eureca-V-gRNA or Eureca-V-sgRNA RNP complex, respectively.

As further illustrated in the Working Examples below, the novel CZE assay of the present disclosure comprises the following steps:
i) Before each run, the capillary is rinsed first with capillary conditioning buffer and then with the separation buffer.
ii) The capillary is filled with the separation buffer maintained at a specific temperature, e.g., 25° C., at all times.
iii) Sample from the sample vial (sample is diluted in CE grade water and stored at a specific temperature, e.g., 4° C.) is injected into the capillary.
iv) The target analytes in the sample are separated according to their charge-to-mass ratios by applying a potential difference along the capillary resulting in an electric field.
v) The separated target analytes are then stained with a fluorescent dye and detected using a laser-induced fluorescence (LIF) detector on a detection window.
vi) The separated target analytes appear as distinct peaks in an electropherogram.
vii) The area under each peak is determined.
viii) The concentration of the target analyte is calculated using its peak area and a standard calibration curve.

In order that this invention may be better understood, the following examples are set forth. These examples are for purposes of illustration only and are not to be construed as limiting the scope of the invention in any manner.

### EXAMPLES

### Example 1: Quantification of Free gRNA Using Capillary Zone Electrophoresis (CZE) Assay

This Example describes the use of a novel capillary zone electrophoresis (CZE) assay described in the present disclosure to quantify free intact gRNA that are not associated with any nuclease in ribonucleoprotein (RNP) products.

### Capillary Zone Electrophoresis (CZE)

The Capillary Zone Electrophoresis (CZE) assay using laser-induced fluorescence (LIF) detection was conducted using a PA 800 Plus Pharmaceutical Analysis System (Sciex, MA). Fluorophores were excited using a 497 nm argon ion laser source (Sciex, MA), whose fluorescence was detected using a 520 nm filter. The data were acquired and analyzed using the 32 Karat software (Sciex, MA). The electrophoresis was performed using a bare fused silica capillary with a total length of 67 cm and an effective length of 50 cm from the point of injection to the detection window. The capillary was pre-rinsed successively with 0.1 N HCl, 0.1 N NaOH, and water at a pressure of 20 psi and for 2 minutes per rinse. The capillary was then rinsed with the separation buffer at a pressure of 20 psi for 4 minutes. The sample (stored at 4° C.) was injected into the capillary at a pressure 20 psi over 5 seconds. The analytes in the sample were separated by applying a 25.1 kV potential difference along the capillary for 15 minutes resulting in an electric field of 424 V cm-1. The capillary was maintained at a temperature of 25° C. at all times. The separation buffer for all analyses was borate buffer, density 1.0000 g/mL, pH 9.2.

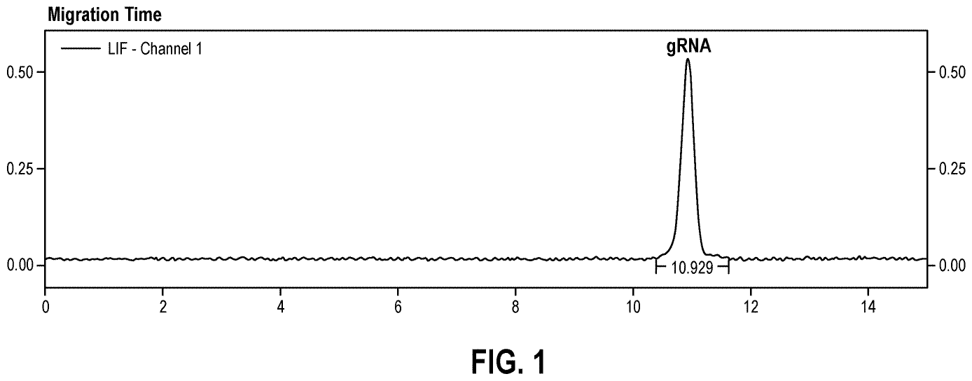

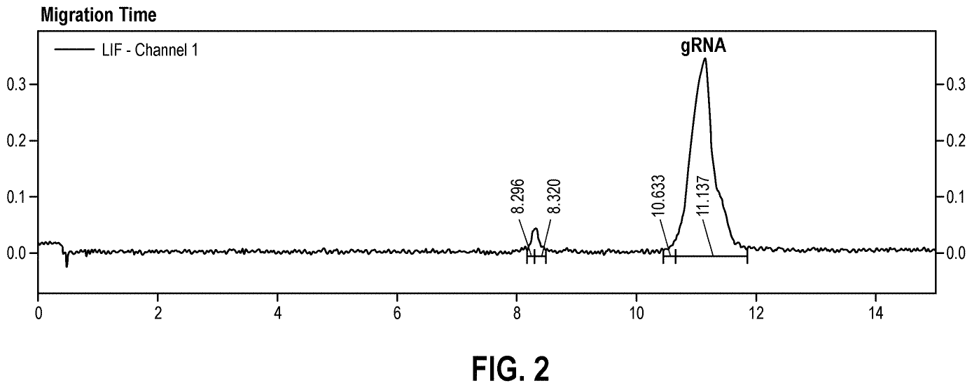

In the CZE assay, separation of the two different sizes of gRNA (40 or 100 nucleotides) from three different nucleases (Streptococcus pyogenes Cas9 (SpCas9); Acidaminococcus Cpf1, and Eureca-V™) took place inside a capillary filled with a separation buffer containing a fluorescent RNA staining dye. The gRNAs were separated from the complexed nucleases and RNPs based on their charge-to-mass ratios and were detected using an LIF detector as indicated by the time taken for the uncomplexed RNAs and RNPs to migrate to the detection window as shown in FIGS. 1 and 2. For each integrated peak in the resulting electropherogram, the 32 Karat Software was used to determine the peak area. The concentration of free gRNA in the RNP sample was calculated by comparing the peak area of the gRNA in the sample with the peak area of the gRNA standard of known concentration using a gRNA standard calibration curve.

### Standard Curve Preparation

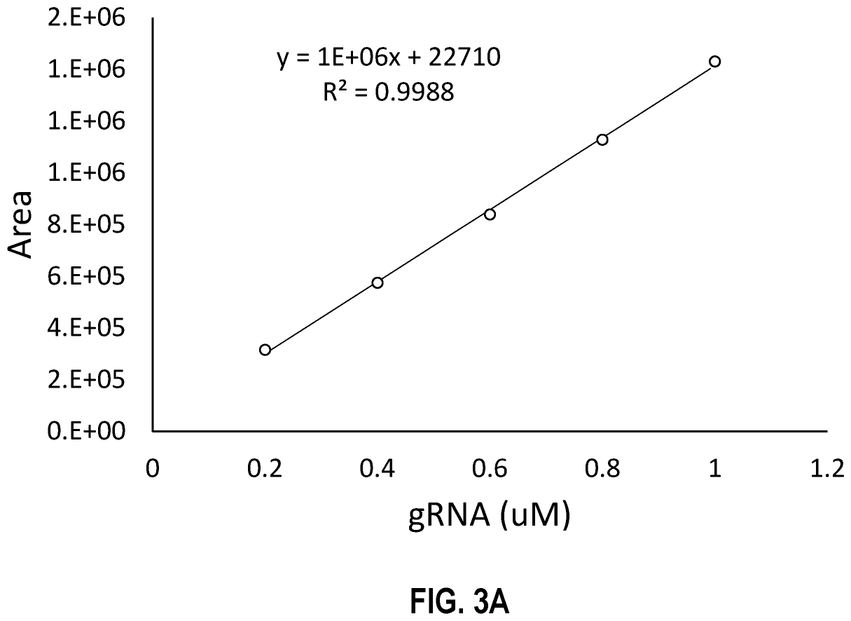

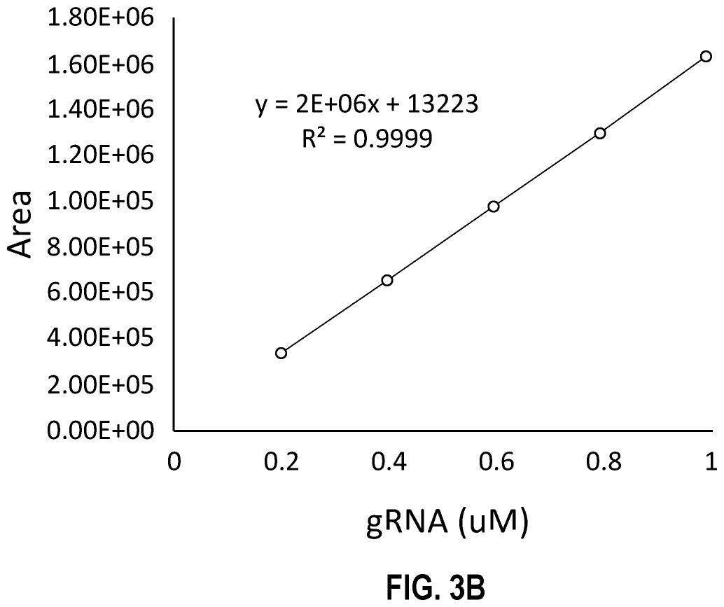

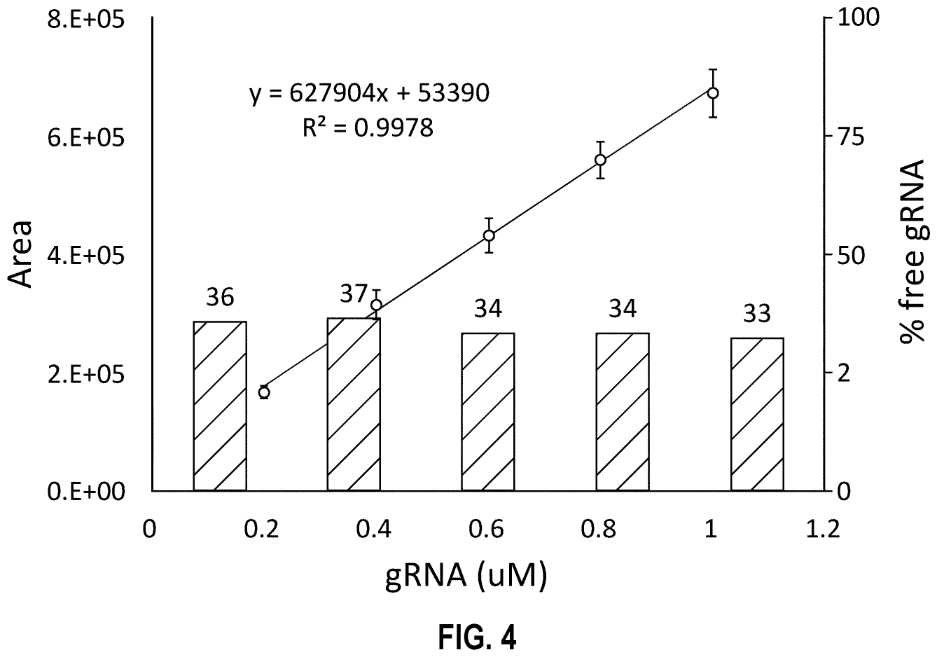

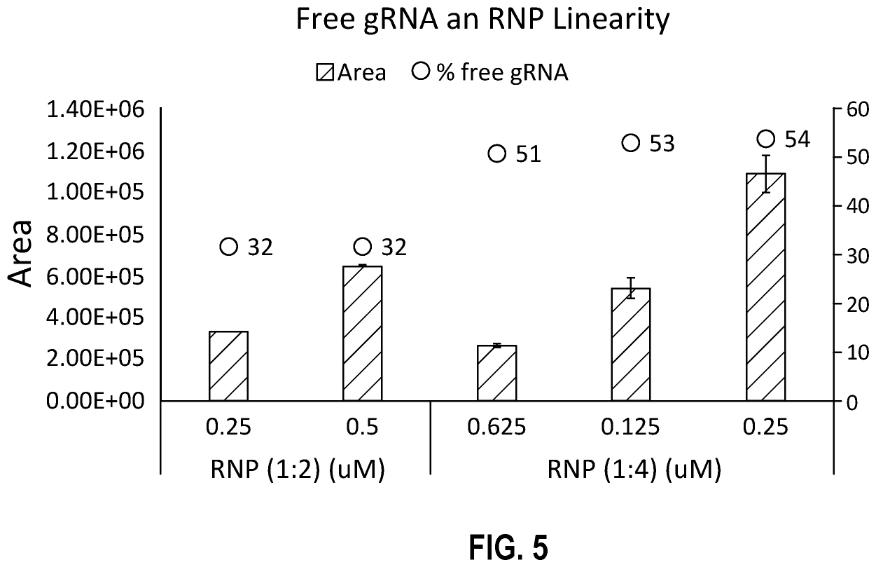

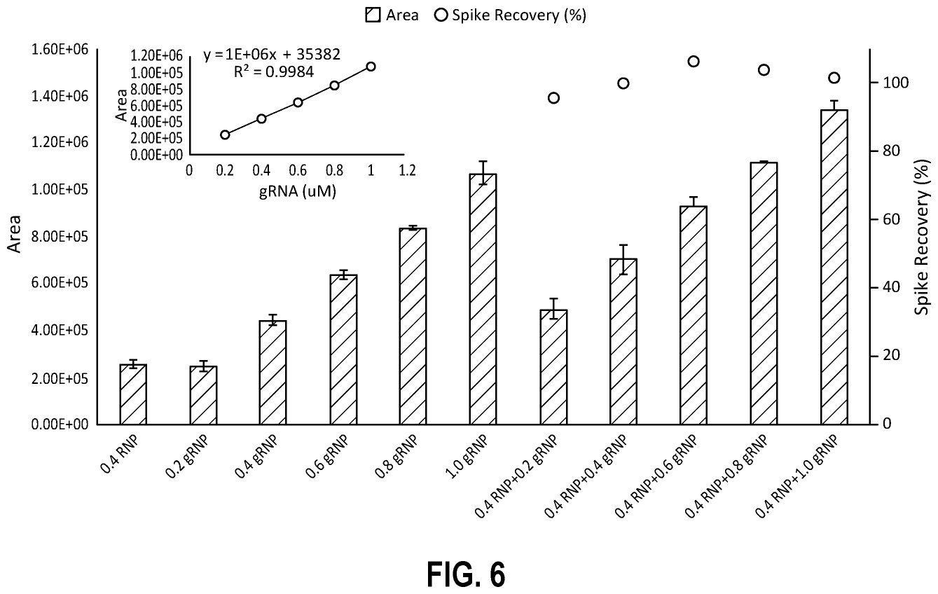

A gRNA (typically same sequence as used in the RNP complex) was diluted to a working concentration of 10 μM with molecular biology grade (MBG) water and mixed well. The gRNA was then serially diluted to final concentrations of 0.2, 0.4, 0.6, 0.8, and 1.0 μM with MBG water and mixed well. The gRNA dilutions were then assayed to plot a standard calibration curve (FIG. 3). The upper and lower ranges of the gRNA concentration used for plotting the standard curve can be altered based on the quality of the gRNA.

TABLE 1
gRNA Standard Curve Preparation

| | | | | Working |
|---|---|---|---|---|
| | 10 μM gRNA | MBG Water | Total Volume | Concentration |
| STD | (μL) | (μL) | (μL) | (μM) |
| 1 | 3 | 147 | 150 | 0.2 |
| 2 | 6 | 144 | 150 | 0.4 |
| 3 | 9 | 141 | 150 | 0.6 |
| 4 | 12 | 138 | 150 | 0.8 |
| 5 | 15 | 135 | 150 | 1.0 |

### Sample Preparation

Each RNP sample was diluted to a final concentration of 1 μM with MBG water and mixed well.

### Separation Buffer

Borate buffer (pH 9.2) was used as the separation buffer.

### Fluorescence Dye

Sybr™ Green II RNA gel stain (Thermo Fisher Scientific) was used as the fluorescent dye.

### Results

In this novel analytic method, the concentration and the percent of unassociated free intact gRNA in an RNP sample was determined. The exemplified method can detect free RNA (e.g., gRNA) at a concentration as low as the lowest reference standard of 0.2 μM or 6.8 μg/ml.

### Example 2: Evaluation of Specificity, Linearity, Accuracy, and Repeatability of the CZE Assay

This Example evaluates specificity, linearity, accuracy, and repeatability of the CZE assay described in Example 1 using RNPs formed at a 3:1 ratio of gRNA to SpCas9. Target acceptance criteria for the parameters being tested are summarized in Table 2 below.

TABLE 2
Study Parameters and Target Criteria

| | | Target Acceptance |
|---|---|---|
| Parameter | | Criteria |
| Linearity | Linear Regression Across | R² ≥ 0.98 |
| | Plot of Measured Values vs. | |
| | Expected Values | |
| Accuracy | Mean % Spike Recovery | 90-110% |
| | for each level | |
| Repeatability | % RSD across 3 results (n = 3) | ≤5% |
| | at each level. | |

### Method

Before conducting the CZE assay, preliminary experiments were run to determine the appropriate gRNA standard curve range, spanning five levels from 0.4 mM to 2.0 mM in 0.4 mM increments. A sixth point at 2.2 mM was added to expand the range of the standard curve. Next, five gRNA spike levels (samples S1 to S5) were selected, ranging from 0.4 mM to 1.2 mM in 0.2 mM increments. An unspiked RNP sample (S0) was analyzed as a control to capture the baseline amount of free gRNA present in the sample. Each sample was then tested as a single injection across three runs to evaluate specificity, repeatability, accuracy, and linearity of the assay method.

### Results

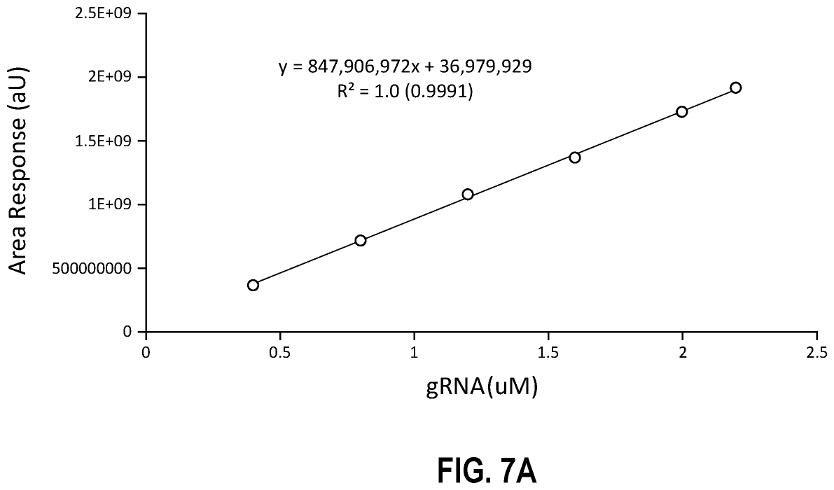

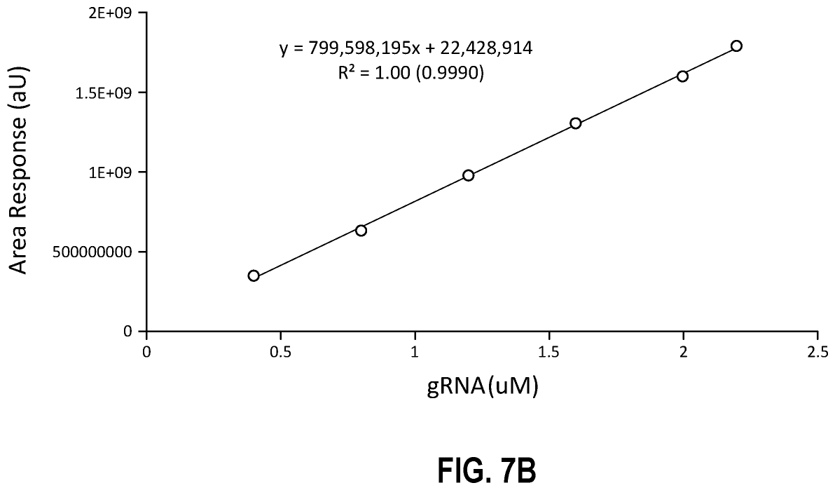

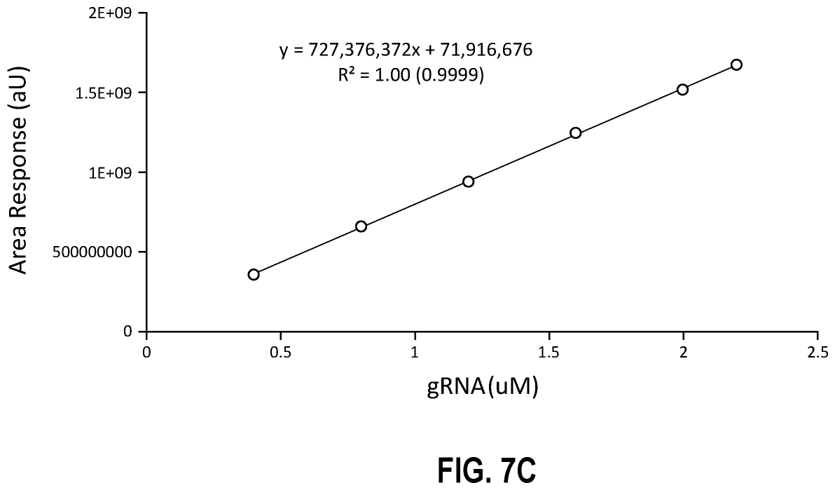

Results across the three runs were respectively analyzed by integrating each result within the instrument software. A linear regression was performed for the six points within each gRNA standard curve (FIGS. 7A-C), which was then used to quantitate the amount of free gRNA present in the unspiked RNP sample and in each of the spiked samples.

### Specificity

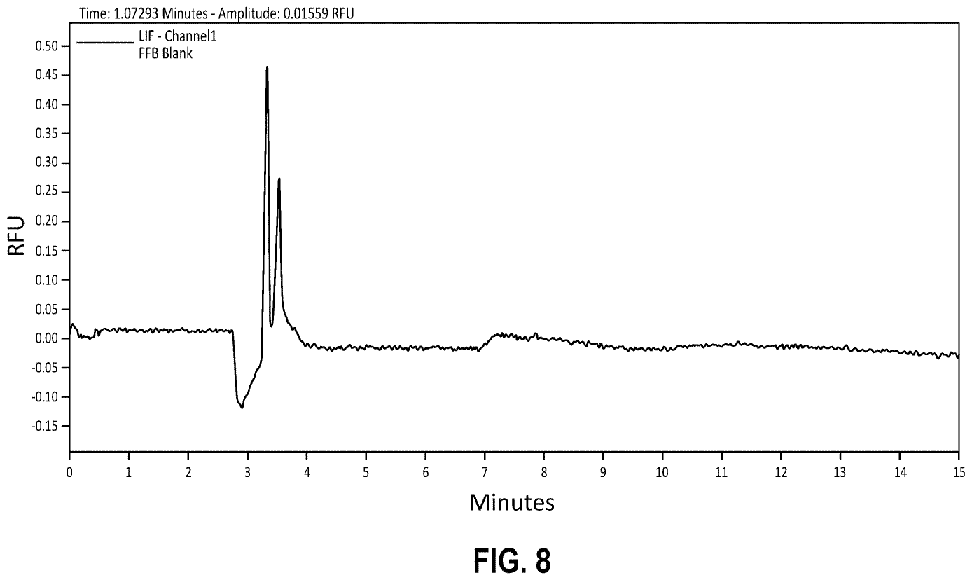

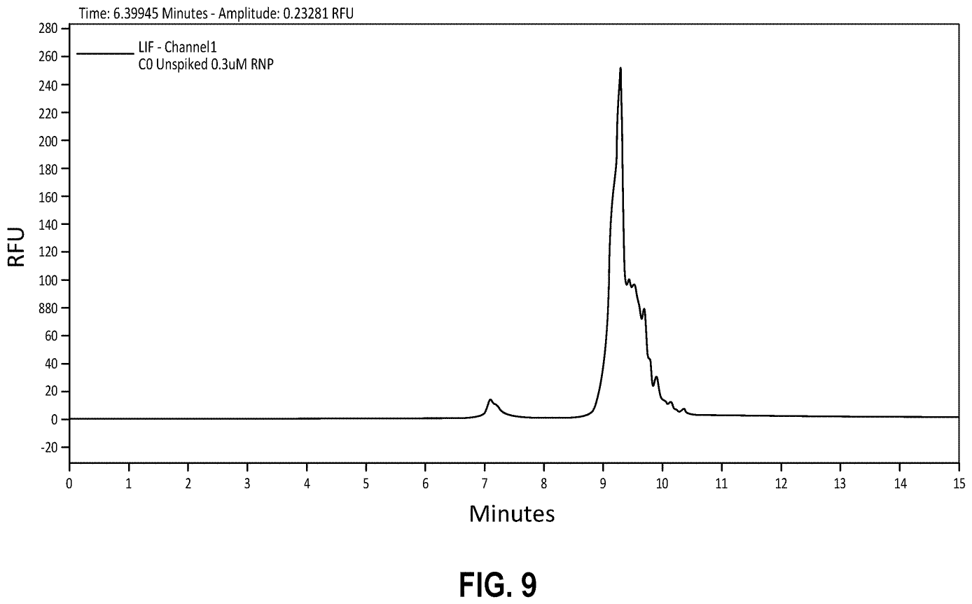

Within each run, the formulation buffer demonstrated non-interference with the free gRNA peak (FIG. 8). The bound gRNA peak is resolved from the free gRNA peak. See FIG. 9 for results.

### Linearity

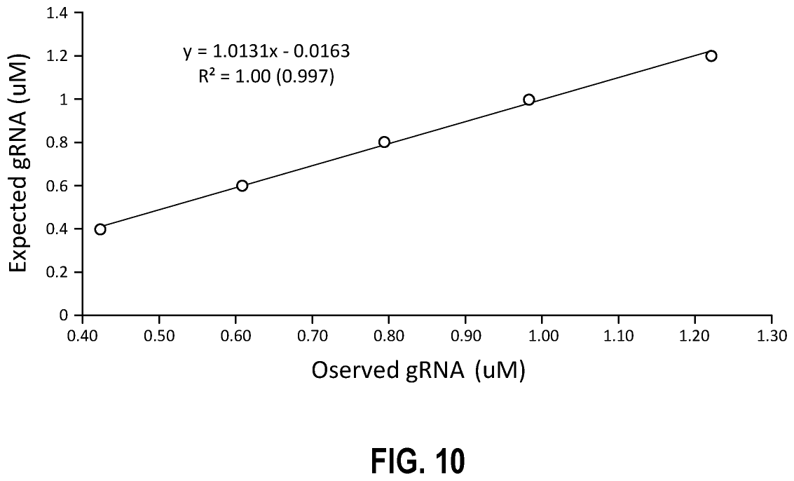

Within each run, the observed quantity of free gRNA in the unspiked RNP sample was subtracted from the total observed result for each spiked sample to quantitate the observed spike quantity at that level. The average observed spike quantity of free gRNA across all three runs was then plotted against the theoretical quantity of free gRNA spiked in at each level and a linear regression was performed. See Table 3 and FIG. 10 for results.

### Accuracy

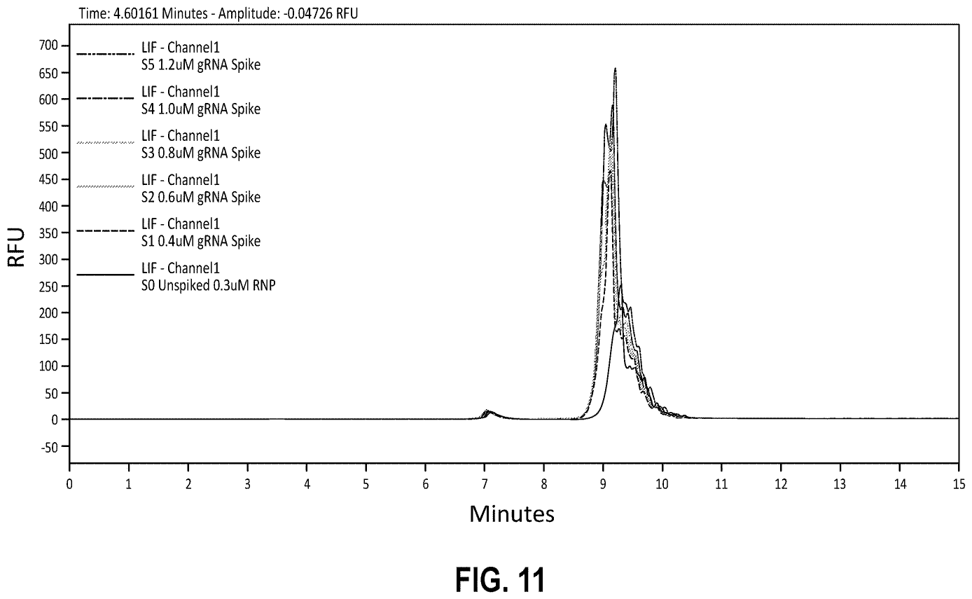

Percent recovery (% recovery) was calculated for each level within each run by taking the ratio of the observed to theoretical spike quantity of free gRNA as a percent. The average percent recovery for each level was evaluated against the initial range criteria of 100±10%. Although one of individual S1 spike levels fell outside of this range, no impact was determined due to the increased sensitivity of this metric at lower levels and the passing result for the average percent recovery at that level. See Table 3 and FIG. 11 for results.

### Repeatability

Repeatability was calculated by taking the percent relative standard deviation (% RSD) across the three runs for the observed total quantity of free gRNA for the unspiked RNP sample and each spiked sample. Results were evaluated against the original target of % RSD≤5%. Due to the relatively small quantities, % RSD for the unspiked RNP sample is more sensitive. For this reason, the % RSD result of the S0 control was accepted against a target of ≤10%. See Table 3 for results.

TABLE 3
Result Summary

| | | Observed | | | Observed | | |
|---|---|---|---|---|---|---|---|
| | Theoretical | Total | Repeatability | | Spike | | |
| Sample | Spike | Run 1-3 | Average | % | Run 1-3 | % Recovery | |
| Level | (mM) | (mM) | (mM) | RSD | (mM) | Run 1-3 | Average |
| S0 | 0.0 | 0.64 | 0.68 | 6.3% | Not | Not | Not |
| Control | | 0.68 | | | Applicable | Applicable | Applicable |
| | | 0.73 | | | | | |
| S1 | 0.4 | 1.04 | 1.11 | 4.8% | 0.41 | 101% | 106% |
| | | 1.13 | | | 0.45 | 113% | |
| | | 1.14 | | | 0.42 | 104% | |
| S2 | 0.6 | 1.24 | 1.29 | 3.4% | 0.60 | 100% | 101% |
| | | 1.31 | | | 0.63 | 105% | |
| | | 1.32 | | | 0.59 | 99% | |
| S3 | 0.8 | 1.42 | 1.48 | 3.6% | 0.78 | 98% | 99% |
| | | 1.48 | | | 0.80 | 100% | |
| | | 1.53 | | | 0.80 | 100% | |
| S4 | 1.0 | 1.62 | 1.66 | 3.3% | 0.98 | 98% | 98% |
| | | 1.72 | | | 1.04 | 104% | |
| | | 1.65 | | | 0.93 | 93% | |
| S5 | 1.2 | 1.86 | 1.90 | 2.1% | 1.22 | 102% | 102% |
| | | 1.94 | | | 1.25 | 105% | |
| | | 1.91 | | | 1.19 | 99% | |

### CONCLUSION

The data above demonstrates that the CZE assay method described in the present disclosure showed remarkable specificity, linearity, accuracy, and repeatability for quantification of free gRNA in the concentration range between 0.2 to 1.0 μM.

1. A method of quantifying by capillary zone electrophoresis (CZE) free RNA present in a composition comprising ribonucleoprotein (RNP) complexes formed between a gene-editing endonuclease and one or more associated RNAs, comprising:
(i) providing a CZE system comprising:
a source reservoir and a destination reservoir containing a separation buffer, wherein the source reservoir further contains an RNA-staining dye,
an anode placed in the source reservoir and a cathode placed in the destination reservoir, wherein the anode and the cathode are connected to a power supply,
a fused silica capillary tube connecting the source reservoir and the destination reservoir, and
a detector proximal to the cathode for detecting signals from the capillary tube,
(ii) introducing the composition to the capillary tube proximal to the anode;
(iii) performing electrophoresis on the system; and
(iv) quantifying signal from RNA-bound dye by using the detector, wherein the quantity of the signal is indicative of the concentration of free RNA in the composition.

2. The method of claim 1, wherein the RNA is a guide RNA (gRNA).

3. The method of claim 1, wherein the gene-editing endonuclease is a CRISPR endonuclease.

4. The method of claim 1, wherein the separation buffer is formulated to maintain the native conformation of the RNP.

5. The method of claim 1, wherein the separation buffer is a borate buffer.

6. The method of claim 1, wherein the RNA-staining dye is a fluorescent dye.

7. The method of claim 6, wherein the fluorescent dye comprises a chromophore unit whose chemical structure is shown below:

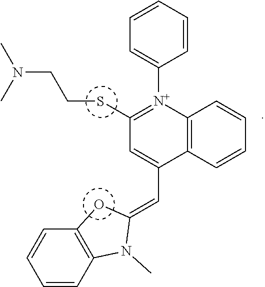 [SMILES: CN(C)CCSc1cc(/C=C2\Oc3ccccc3N2C)c2ccccc2[n+]1-c1ccccc1]

8. The method of claim 1, wherein the detector is a laser-induced fluorescence (LIF) detector.

9. The method of claim 2, wherein the gRNA is at least 40 or at least 100 nucleotides in length.

10. The method of claim 3, wherein the CRISPR endonuclease is selected from Streptococcus pyogenes Cas9 (SpCas9), Acidaminococcus Cpf 1, and Eureca-V.

11. The method of claim 1, wherein free RNA can be detected at a concentration as low as 0.2 pM or 6.8 pg/ml.

12. A composition comprising a ribonucleoprotein (RNP) complex formed between a gene editing endonuclease and one or more associated RNAs, said composition obtained by a process that separates any free RNA from the RNP complex, wherein the process comprises:
(i) providing a CZE system comprising:
a source reservoir and a destination reservoir containing a separation buffer, wherein the source reservoir further contains an RNA-staining dye,
an anode placed in the source reservoir and a cathode placed in the destination reservoir, wherein the anode and the cathode are connected to a power supply,
a fused silica capillary tube connecting the source reservoir and the destination reservoir,
and
a detector proximal to the cathode for detecting signals from the capillary tube,
(ii) introducing the composition to the capillary tube proximal to the anode;
(iii) performing electrophoresis on the system to separate the free RNA from the RNP complex; and
(iv) quantifying signal from RNA-bound dye by using the detector, wherein the quantity of the signal is indicative of the concentration of free RNA in the RNP complex.

13. The composition of claim 12, wherein the RNA is a guide RNA (gRNA).

14. The composition of claim 12, wherein the gene-editing endonuclease is a CRISPR endonuclease.

15. The composition of claim 12, wherein the separation buffer is formulated to maintain the native conformation of the RNP.

16. The composition of claim 12, wherein the separation buffer is a borate buffer.

17. The composition of claim 12, wherein the RNA-staining dye is a fluorescent dye.

18. The composition of claim 17, wherein the fluorescent dye comprises a chromophore unit whose chemical structure is shown below:

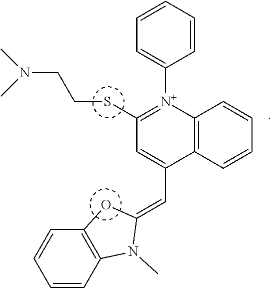 [SMILES: CN(C)CCSc1cc(/C=C2\Oc3ccccc3N2C)c2ccccc2[n+]1-c1ccccc1]

19. The composition of claim 12, wherein the detector is a laser-induced fluorescence (LIF) detector.

20. The composition of claim 13, wherein the gRNA is at least 40 or at least 100 nucleotides in length.

21. The composition of claim 14, wherein the CRISPR endonuclease is selected from Streptococcus pyogenes Cas9 (SpCas9), Acidaminococcus Cpf1, and Eureca-V.

22. The composition of claim 13, wherein the concentration of free RNA is at least than 0.2 pM or 6.8 pg/ml.
